(19) United States
(12) Patent Application Publication
(10) Pub. No.: US 2012/0079899 A1
(43) Pub. Date: Apr. 5, 2012

(54) PHYSIOLOGICAL SIGNAL SENSING DEVICE, CONTAINING DEVICE AND METHOD FOR WEARING A PROTECTIVE FILM

(75) Inventors: Chun Liang LU, New Taipei City (TW); Chih Hsien Huang, New Taipei City (TW); Yi-Hsin Huang, New Taipel City (TW); Chao-Wang Chen, Taipei City (TW)

(73) Assignee: TAIDOC TECHNOLOGY CORPORATION

(21) Appl. No.: 13/249,222

(22) Filed: Sep. 29, 2011

(30) Foreign Application Priority Data
Oct. 5, 2010 (TW) 099133943

(51) Int. Cl. G01N 33/00; B65D 85/00

(52) U.S. Cl. 73/865.8; 206/524.1

(57) ABSTRACT

A physiological signal sensing device, a containing device and a method for wearing a protective film are provided. The physiological signal sensing device includes a housing, a sensing module, a fastening, and a transporter. The housing includes an opening. The sensing module is engaged within the housing and used for sensing vital signal(s). The transporter is engaged within the housing and transports a protective film. When the sensing module is moved to a predetermined site, the fastening fixes an area of the protective film at the opening. When the sensing module keeps being moved through the opening, the area of the protective film is stretched over the sensing module so as to properly fix the area of the protective film to cover the sensing module evenly.

### BACKGROUND OF THE INVENTION

1. Field of the Invention

The present invention relates to a device for properly fixing a protective film, and particularly relates to a containing device or a physiological signal sensing device with a fastening to fix a protective film.

2. Description of the Related Art

Kinds of electrical thermometer have been developed such as a pen-shaped body thermometer, a pacifier-shaped thermometer, or an IR thermometer.

Before using an ear thermometer such as IR ear thermometer, users often use a protective cap, such as a cone-shaped one, to cover a probe of the ear thermometer to provide hygienic protection. After covering a cap over the probe head, user put the probe into the ear canal of the one who are being measured to receive infrared radiation and then a signal indicating the patient's body temperature is shown. After measuring, the used protective cap will be thrown away. Then, before the next measurement, a new cap will be used to cover the probe for the next people who are being measured to prevent cross infection by directing contacting the probe.

However it is still risky to contact polluted sources by directly touching the used protective caps by hand.

Refer to FIG. 1, FIG. 1 is an ear thermometer with a protective-cap cassette in prior art. The protective-cap cassette (91) is used for containing few protective caps (910) and engaged within a handle portion (901) of a housing. The protective cap (910) comprises a solid plastic base (912) to hold a film (913) which can be extended.

However, the volume of the cassette (91) and protective caps (910) are large and they are made with more complicate procedures. Whereby this kind of ear thermometer can only contain few caps but the size is pretty large resulting in many inconveniences while using.

### SUMMARY OF THE INVENTION

In order to solve the above noted conventional problems, one aspect of the present invention is to provide a physiological signal sensing device, a containing device and a containing device for containing protective film that achieve for properly fixed and stretched the protective film over a sensing module or a container.

The present invention relates to a physiological signal sensing device for a protective film to be properly fixed and stretched over a sensing module.

The present invention relates to a containing device for a protective film to be properly fixed and stretched over a container.

The present invention relates to a containing device for containing a protective film, which is adapted for the containing device or the physiological signal sensing device described above.

The present invention relates to a method for wearing a protective film, which is adapted for the containing device or the physiological signal sensing device described above.

In an aspect of the present invention, a physiological signal sensing device is provided. The physiological signal sensing device comprises a housing, a sensing module, a transporter, and a fastening. The housing includes an opening. The sensing module is engaged with the housing and used for sensing a vital signal(s). The transporter is engaged within the housing and transports an area of a protective film to a site of a moving path which the sensing module is moved toward the opening, and the protective film has multiple areas. The fastening is engaged within the housing and used for fixing the area at the opening. When the sensing module is moved to a predetermined site, the fastening fixes the area of the protective film at the opening. When the sensing module keeps being moved through the opening, the area of the protective film is stretched over the sensing module so as to properly fix the area of the protective film and evenly cover the sensing module.

In an embodiment of the present invention, the physiological signal sensing device further includes a resistant. The resistant is engaged with the sensing module. When the sensing module is moved to a predetermined site, the resistant is driven by the sensing module so as to form a resisting force against the motion of the sensing module.

In an embodiment of the present invention, the physiological signal sensing device further includes an immobilizing assembly. The immobilizing assembly is connected between the sensing module and the housing. After the area has been stretched over the sensing module, the immobilizing assembly is fixed at a predetermined position so as to maintain a situation of the area being stretched over the sensing module.

The immobilizing assembly may be designed as a coupling assembly with a male member and a female member such as a protrusion and a corresponding depression disposed separately at the sensing module and a predetermined site of the housing.

In an embodiment of the present invention, the physiological signal sensing device further includes a controller. The controller disposed at said sensing module and extended out of the housing for providing a holding area for moving said sensing module.

In an embodiment of the present invention, the physiological signal sensing device further includes a protective-film containing assembly. The protective-film containing assembly is engaged with the transporter for containing the protective film.

In an embodiment of the present invention, the physiological signal sensing device further includes a following. The following is connected between the sensing module and the fastening and driven by the motion of the sensing apparatus to drive the fastening to operate.

In an embodiment of the present invention, the physiological signal sensing device further comprises a driving assembly. The driving assembly is connected between the sensing module and the transporter, wherein, the driving assembly is driven by the sensing module to drive the transporting device to transport the protective film. The driving assembly can be designed in any types as needed such as gear assembly or wheel-shaft assembly.

In an embodiment of the present invention, the physiological signal sensing device further comprises a follower. The follower is connected between the sensing module and the driving assembly for have the driving assembly be driven by the motion of the sensing apparatus.

In another aspect of the present invention, a containing device is provided. The containing device comprises a housing, a container, a transporter and a fastening. The housing includes an opening. The container is engaged within said housing. The transporter is engaged within the housing for transporting an area of a protective film to a site of a moving path which the container moves toward the opening, and the protective film has multiple areas. The fastening is engaged within the housing and used for fixing the area at the opening, and the opening is adapted for the container to move through; when the container is moved toward the opening to a predetermined site, the fastening fixes the area at the opening; when the container keeps being moved through the opening, the area is stretched over the container.

The containing device can contain any apparatus that can be engaged within the container, such as a sensing module, or a hard structure member depending on the design.

In an embodiment of the present invention, the containing device further comprises a resistant. The resistant is engaged with the container. When the container is moved to a predetermined site, the resistant is driven by the container so as to form a resisting force against the motion of the container.

In an embodiment of present invention, the containing device further includes an immobilizing assembly. The immobilizing assembly is connected between the container and the housing. After the area has been stretched over the container, the immobilizing assembly is fixed at a predetermined position so as to maintain a situation that the area being stretched over the container.

In an embodiment of the present invention, the containing device further includes a protective-film containing assembly for containing the protective-film. The protective-film containing assembly is engaged with the transporter.

In an embodiment of the present invention, the containing device further includes a following. The following is connected between the container and the fastening and driven by the motion of the container to drive the fastening.

In an embodiment of the present invention, the containing device further comprises a driving assembly. The driving assembly is connected between the container and the transporter, and the driving assembly is driven by the container to drive the transporting device to transport the protective film. The driving assembly can be designed as needed such as gear assembly or wheel-shaft assembly.

In an embodiment of the present invention, the containing device further comprises a follower. The follower is connected between the container and the driving assembly for having the driving assembly be driven by the motion of the container.

In an embodiment of the present invention, the sensing module can be designed as needed such as detachable type or fixed type engaged in the physiological signal sensing device or the containing device.

In an embodiment of the present invention, the transporter can be design as needed such as detachable type or fixed type engaged in the physiological signal sensing device or the containing device.

In one another aspect of the present invention, a containing device is provided. The containing device is used for containing a protective film. The containing device is adapted for a physiological signal sensing device. The physiological signal sensing device comprises a housing, a sensing module, a transporter and a fastening, and said housing includes an opening, and the sensing module, the transporter and the fastening are engaged within the housing. The containing device comprises a containing portion for containing a protective film. The containing portion is engaged with the transporter, and the protective film has multiple areas. The transporter is used for transporting an area of the protective film to a site of the moving path that the sensing module moves toward the opening; when the sensing module is moved toward the opening to a predetermined site, the fastening fixes the area at the opening. When the sensing apparatus keeps being moved through said opening, the area is stretched over said sensing module.

In an embodiment of the present invention, the containing device for containing a protective film further includes an accepting portion for accepting the protective film from the containing portion.

In yet another aspect of the present invention, a method for wearing a protective film is provided. The method is adapted for a physiological signal sensing device comprising a housing, a sensing module, a transporter and a fastening. The housing includes an opening. The sensing module used for sensing vital signal(s), and the transporter and the fastening are engaged within the housing.

The method comprises transporting a first area of a protective film to a site of a moving path that the sensing module moves toward the opening by the transporter, and the protective film has multiple areas; then, fixing the first area at the opening by the fastening; moving the sensing module through the opening so as to have the first area stretched over the sensing module; in addition, transporting a second area of said protective-film to the position of the first area located to replace the first area by the transporter; then, fixing the second area at the opening by the fastening; and moving the sensing module through the opening so as to have the second area stretched over the sensing module.

In an embodiment of present invention, the resistant can be spring, damper or those providing a resisting force.

In an embodiment of present invention, the sensing module is designed for sensing vital signal. The vital signal can be such as body temperature, forehead temperature, ear temperature, dryness of skin or sonographic image signals.

In an embodiment of the present invention, the protective film has good tractility and stretchable. It can be made of such as polyethylene, polyvinyl chloride or other material with good tractility.

In an embodiment of the present invention, the fastening is used for fixing an area of a protective film at the opening, and it can be designed as a hard structure or preferably an elastic structure.

Preferably, the elastic structure is a spring structure disposed surround the sensing module. The spring can change the displacement energy of the sensing module or the container into elastic potential energy for providing a resisting force against the motion of the sensing module or the container.

As described above, the present invention provides a fastening for properly fixing a protective film and making an area of the protective film be evenly stretched over a sensing module or a container; whereby user can prevent contacting the protective film directly and prevent the interference of measurement due to unevenness of the protective film while it is stretched.

More exemplary embodiments and advantages of the present invention may be ascertained by reviewing the present disclosure and the accompanying drawing. It is intended that the description and embodiments with reference to the accompanying drawing to be considered as exemplary only.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a view of a disclosed ear thermometer and a protective cap cassette thereof in the prior art;

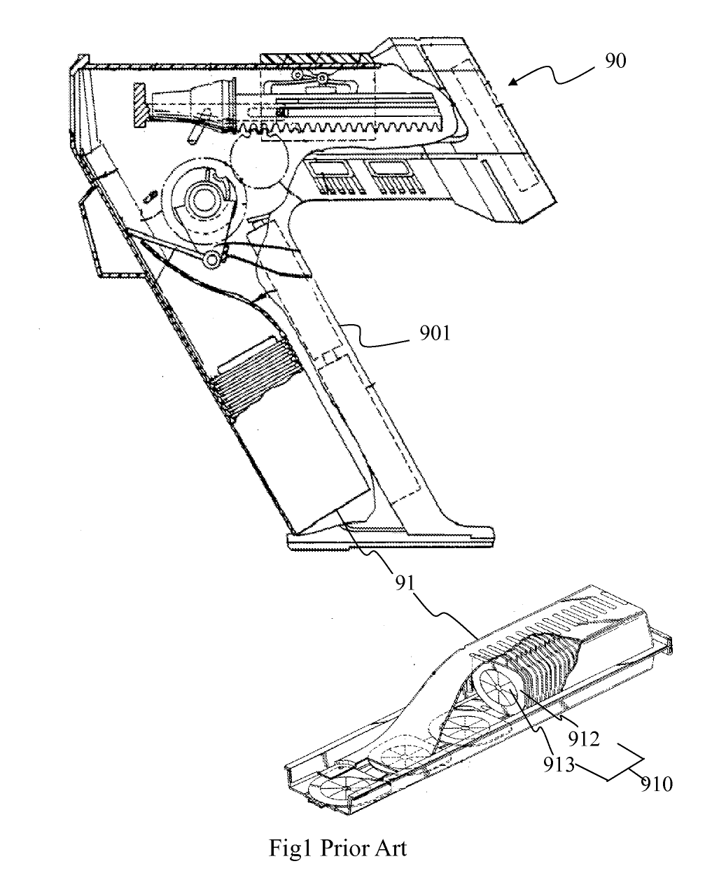

FIG. 2A is a side perspective view of a first embodiment of a partial physiological signal sensing device in accordance with the present invention;

FIG. 2B is a side perspective view of the partial physiological signal sensing device referring to FIG. 2A showing that a sensing module being moved toward an opening till the sensing module disconnects with a driving assembly;

FIG. 2C is a side perspective view of a partial physiological signal sensing device referring to FIG. 2A showing that the sensing module being moved over a predetermined site;

FIG. 2D is a side perspective view of a partial physiological signal sensing device referring to FIG. 2A showing that the sensing module is moved rearwards away from the opening to connect with the driving assembly;

FIG. 3A is a schematic structure view of a partial physiological signal sensing device referring to FIG. 2A showing that the sensing module being moved toward a predetermined site;

FIG. 3B is a schematic structure view of a partial physiological signal sensing device referring to FIG. 2A showing that the sensing module being moved at the predetermined site;

FIG. 3C is a schematic structure view of a partial physiological signal sensing device referring to FIG. 2A showing that the sensing module being moved over the predetermined site;

FIG. 3D is a schematic structure view of a partial physiological signal sensing device referring to FIG. 2A showing that the sensing module is moved rearwards away from the opening;

FIG. 4 is a flow chart of a method for wearing a protective film of a physiological signal sensing device in accordance with the present invention;

FIG. 5 is a schematic structure view of the second embodiment of a partial physiological signal sensing device in accordance with the present invention;

FIG. 6A is a schematic structure view of a third embodiment of a partial physiological signal sensing device in accordance with the present invention showing that the sensing module is moved before a predetermined site;

FIG. 6B is a schematic structure view of a partial physiological signal sensing device referring to FIG. 6A that the sensing module is moved over the predetermined site;

FIG. 7 is a side perspective view of the forth embodiment of a partial physiological signal sensing device in accordance with the present invention; and

FIG. 8 is an enlargement view of a protective-film containing assembly referring to FIG. 7.

### DESCRIPTION OF THE PREFERRED EMBODIMENTS

For the purposes of promoting an understanding of the principles of the invention, reference will now be made to the embodiment illustrated in the drawings, and specific language will be used to describe that embodiment. It will nevertheless be understood that no limitation of the scope of the invention is intended. Alterations and modifications in the illustrated device, and further applications of the principles of the invention as illustrated therein, as would normally occur to one skilled in the art to which the invention relates are contemplated, are desired to be protected. Such alternative embodiments require certain adaptations to the embodiments discussed herein that would be obvious to those skilled in the art.

The following description and accompanying drawings are some examples in accordance with the present invention. The same symbol herein in the drawings indicates the same or similar structure.

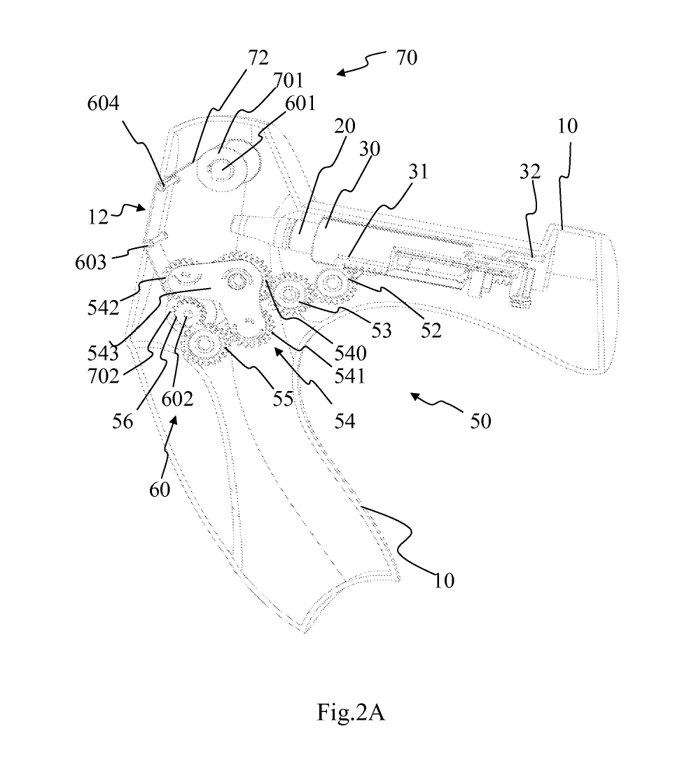

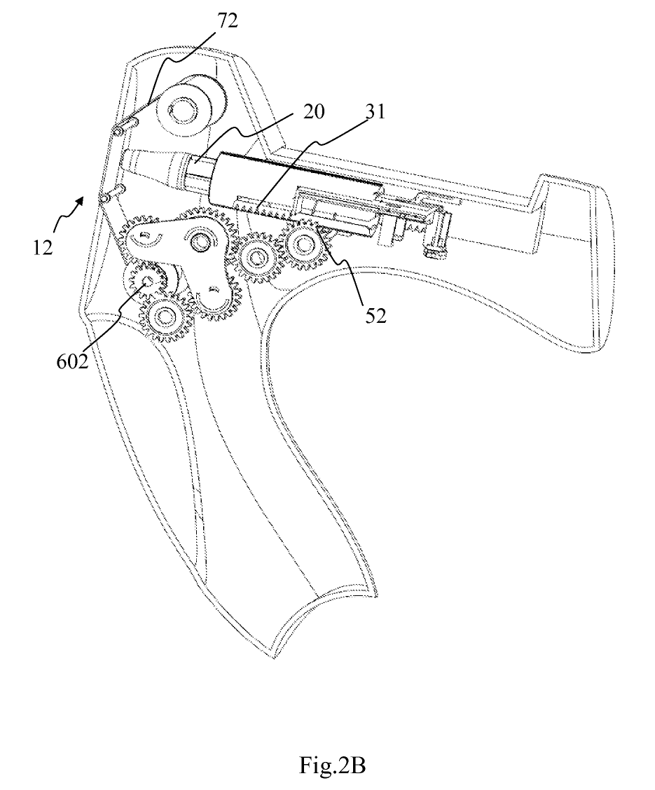

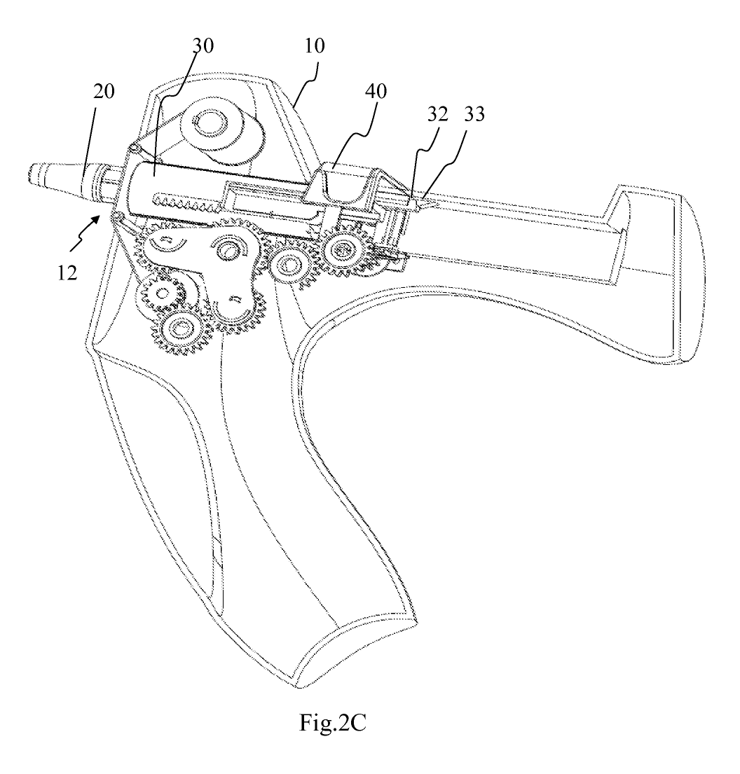

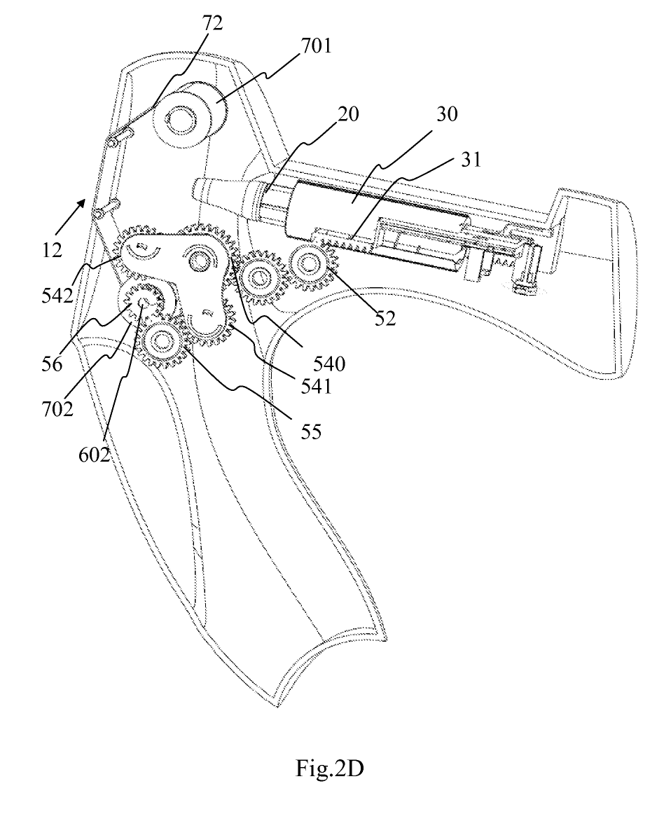

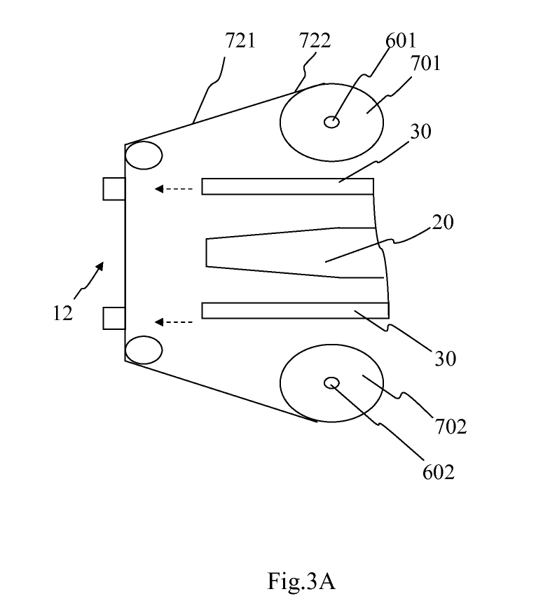

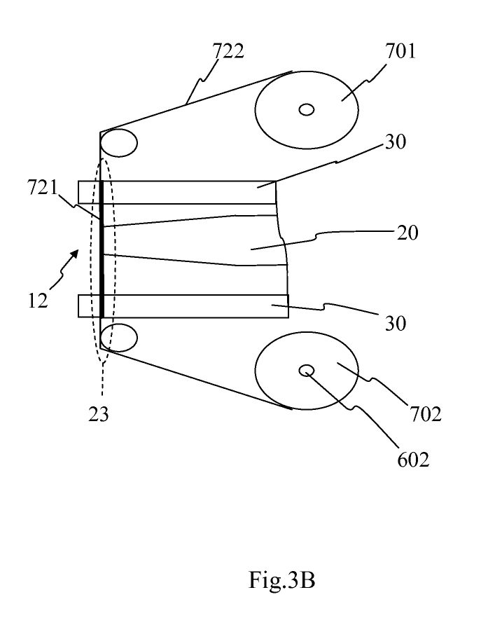

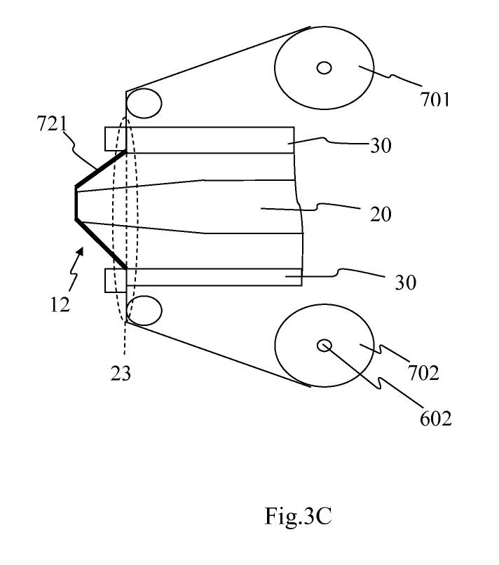

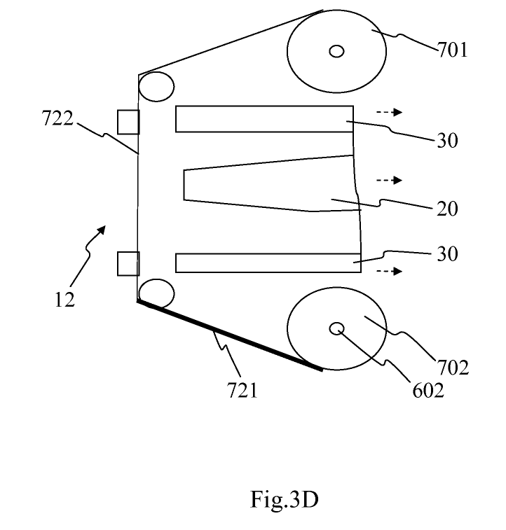

A first embodiment of the present invention refers to FIGS. 2A to 2D, and FIGS. 3A to 3D.

FIG. 2A is a partial side perspective view of the first embodiment of a physiological signal sensing device in accordance with the present invention. FIG. 2C is a side perspective view showing that a sensing module of the physiological signal in FIG. 2A being moved across a predetermined site.

Please refer to both FIGS. 2A and 2C, the physiological signal sensing device comprises a housing (10), a sensing module (20), a fastening (30), a controller (40), a driving assembly (50), a transporter (60), a protective-film containing assembly (70) and a protective film (72). FIG. 2A ignores some parts of the fastening (30) and controller (40) at front end surrounding the sensing module (20) in order to mark the location of the sensing module (20) and so does in FIGS. 2B and 2D.

Please refer to FIG. 2A. The housing (10) includes an opening (12). The sensing module (20) herein is an ear thermometer engaged within the housing (10).

The fastening (30), a follower (31) and the controller (40) are disposed at outer part of the sensing module (20)

Please further referring to FIG. 2C first, a part of the controller (40) is disposed outside the housing (10) and another part of the controller (40) mounted into the housing (10) to connect with the sensing module (20) for providing a holding site. The controller (40) is moved along a hollow channel formed alongside the housing (10) and is extended from a side of the sensing module (20) and out of the housing (10). Whereby, users are able to move the sensing module (20) by moving the controller (40) along the channel. The controller (40) can also be designed other structure as needed, such as in another embodiment that the controller (40) can be designed as an extending part from a back end of the sensing module (20) and extended out from the back end of the housing (10) to provide a site to press.

Now referring back to FIG. 2A, the sensing module (20) is surrounded by the fastening (30). More specifically, the fastening (30) is movably engaged with the sensing module (20) by a connector. The connector in this embodiment of the present invention is a spring. In other embodiments of the present invention, it can also be connected by any selected connector as needed, such as a gear or wheel. When the sensing module (20) is moved forward to the opening (12), the fastening (30) is driven by the sensing module (20) to move toward the opening (20) so as to be able to fix an area of the protective film (72) at the opening (12).

The follower (31) is disposed at an outer site relative to the sensing module (20). More specifically, the follower (31) is disposed at one side of the fastening (30) and indirectly connects the sensing device (20) and the driving assembly (50).

Please now referring to both FIGS. 2A and 2C, the physiological signal sensing device of the embodiment in accordance with the present invention further includes an immobilizing assembly. The immobilizing assembly can be designed as a coupling assembly such as an assembly with a protrusion and a corresponding depression. In this embodiment, the immobilizing assembly is designed as two protrusions (32, 33). The protrusion (32), shown in FIG. 2A, is disposed on the sensing module (20). Another protrusion (33), shown in FIG. 2C, is disposed at an inner side of the housing (10) at the moving path that the sensing module (20) moved along.

Now referring back to FIG. 2A, the driving assembly (50) of the embodiment is a gear assembly including gears (52, 53, 54, 55 and 56). The gear (52) is able to connect with the follower (31) so as to be driven by the motion of the sensing module. The gear (54) comprises connecting gears (540, 541, and 542) and a connecting member (543). The connecting member (543) connects each axle center of connecting gears (540, 541, 542) so as to form gear (54) which can be changed in its connection situation by operation. The gear (53) is connected between the gear (52) and the connecting gear (540). By the motion forward to the opening or rearward away from the opening of the sensing module (20) can the connecting gear (541) be indirectly driven to engage with the gear (55), or selectively can the connecting gear (542) be indirectly driven to engage with the gear (56). While the connecting gear (541) is engaged with the gear (55), the connecting gear (542) is disengaged with the gear (56). In contrast, while the connecting gear (541) is disengaged with the gear (55), the connecting gear (542) engaged with the gear (56). Whereby, the driving assembly (50) is switchable to drive the transporter (60) to move.

The transporter (60) may comprise one or more transporting members for transporting the protective film (72). In this embodiment, the transporter comprises transporting members (601, 602, 603, and 604) and each of them is a columned rotatable structure. The protective film (72) can be transported by the rotation of transporting members (601, 602, 603, and 604).

The protective-film containing assembly (70) includes a containing portion (701) and an accepting portion (702). The transporting members (601, 602) respectively connect with the containing portion (701) and the accepting portion (702). The transporting members (603, 604) are respectively disposed at two sides of the opening (12). The accepting portion (702) can be rotated by the rotation of the transporting member (602). The rotation of the accepting portion (702) can drive the protective film (72) to move so as to drive the transporting members (601, 603, 604) to rotate and whereby the transporting members (601, 603, 604) transport the protective film (72) to pass across the opening (12) in a direction from the containing portion (701) toward the accepting portion (702).

To describe more specific about the operation process of the first embodiment in accordance with the present invention, please refer to both FIGS. 2A and 3A first. FIG. 3A is a schematic structure view of a portion of the physiological signal sensing device referring to FIG. 2A showing that the sensing module being moved toward a predetermined site. Please first refer to FIG. 3A, when the sensing module (20) is moved toward to the opening (12), the fastening (30) is also moved toward the opening (12) following with the sensing module (20). Then refer to FIG. 2A, after the sensing module (20) is moved till the follower (31) connects with the gear (52), the motion of the sensing module (20) drives a series action process of the gear set. First, the gear (52) rotates in counterclockwise direction so as to indirectly drive the connecting gear (540) to rotate in counterclockwise direction. The force of the rotation of the connecting gear (540) can drive the connecting member (543) to rotate in counterclockwise direction so as to have the connecting part which connects the connecting member (543) and the connecting gear (542) to move downward so as to have the connecting gear (542) connect the gear (56) and have the connecting gear (541) separated from the gear (55). By keeping rotating the gear (540) will drive the connecting gear (542) to rotate in clockwise direction so as to drive the gear (56) and the transporting member (602) to rotate in counterclockwise direction. Whereby, the transporter (60) can transports the protective film which has multiple areas includes area (721) and area (722) from the containing portion (701) to the accepting portion (702).

Then, pleas refer to FIG. 2B. FIG. 2B is a side perspective view of a partial physiological signal sensing device referring to FIG. 2A showing that the sensing module being moved toward an opening till the sensing module disconnects with the driving assembly.

After the sensing module (20) keeps being moved toward the opening (12) till the follower (31) disconnect with the gear (52) as FIG. 2B shown, the transport member (602) won't be driven by the sensing module (20) so as not to transport the protective film. As a result, the protective film stops moving. Then refer to FIG. 3B, FIG. 3B is a schematic structure view of a partial physiological signal sensing device referring to FIG. 2A showing the sensing module being moved to the predetermined site. When the sensing module (20) keeps being moved to a predetermined site (23) showed in dotted line, the fastening (30) is driven by the sensing module (20) to move to the predetermined site (23) so as to fix the area (721) at the opening (12).

Please refer to FIG. 3C, FIG. 3C is a schematic structure view of a partial physiological signal sensing device referring to FIG. 2A that the sensing module being moved over the predetermined site. When the sensing module (20) keeps being moved over the predetermined site (23) and passing through the opening (12), the area (721) is fixed and stretched over the sensing module (20) by the force from the fastening (30) and the sensing module (20). Due to the area (721) evenly covering the sensing module (20), the interference of temperature sensing from the protective film can be decreased.

Refer to FIG. 2C, after a part of the protect film (72) is stretched over the sensing module (20) (figure not shown) by moving the sensing module (20), the protrusion (32) is also moved with the motion of the sensing module (20) to couple with the protrusion (33), and therefore the sensing module can be immobilized. Therefore, user does not need to provide force to maintain the situation that the protective film covering over the sensing module. Whereby, it can increase convenience while using. When the protrusion (32) is decoupled from the protrusion (33), then it may have the sensing module (20) turn back as a movable one.

Please now refer to both FIGS. 2D and 3D. FIG. 2D is a side perspective view of the physiological signal sensing device referring to FIG. 2A showing that the sensing module is moved rearwards away from the opening to connect with the driving assembly. FIG. 3D is a schematic structure view of a partial physiological signal sensing device referring to FIG. 2A that the sensing module is moved rearwards away from the opening.

Please refer to FIG. 3D first, when the sensing module (20) is moved rearward away from the opening (as arrow shown), the fastening (30) is also moved rearward following with the motion rearward of the sensing module (20).

Refer to FIG. 2D, when the sensing module (20) is move rearwards till the follower (31) connects with the gear (52) again and then keeps being moving rearwards so as to drive the gear set with series action.

The gear (52) is driven by the motion of the follower (31) to rotate in clockwise direction. The rotation of the gear (52) indirectly drives the connecting gear (540) to rotate in the same direction. The rotation force have the connecting member (543) to rotate in clockwise so as to have the portion which connect the connecting member (543) and the connecting gear (541) to move a little bit to the left. Whereby, the connecting gear (541) can connect with the gear (55) and the connecting gear (542) disconnect with the gear (56).

While the sensing module (20) keeps being moved, the gear (55) will be driven to rotate in clockwise direction. The rotation of the gear (55) drives the gear (56) to rotate in counterclockwise direction so as to drive the transporting member (602) to rotate in counterclockwise direction. Whereby, the transporter (60) transports the protective film (72) from the containing portion (701) to the accepting portion (702).

By means of the acting process described above referring to FIG. 2D, the area (722) referring in FIG. 3D is transported to the moving path which the sensing module (20) is moved toward the opening (12) and replace the area (721). The area (721) then is transported toward the accepting portion (702). By means of the driving assembly (50), whether the sensing module (20) is moved forward or backward or not, the protective film (72) can be transported. Hence, by the time the used area of protective film (72) is transported away from the opening (12), a new one is transported to the opening (12) in order to not only avoid using the used area by user but also efficiently use the moving distance of the sensing module (20) to move an area of an unused protective film to the opening (12).

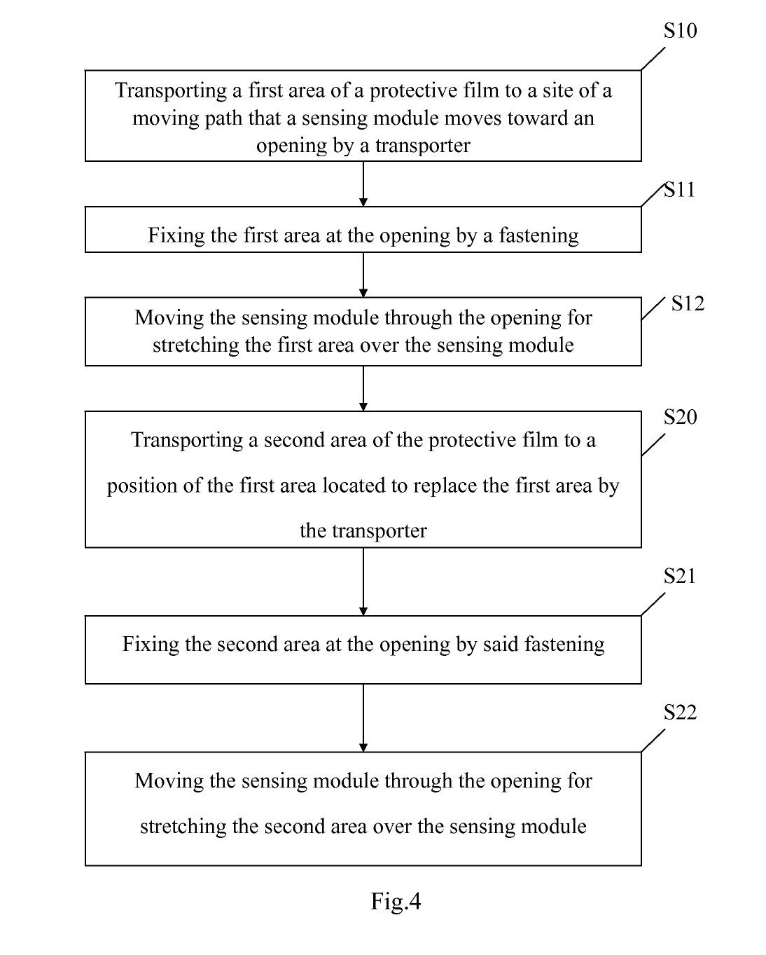

In addition, please refer to FIGS. 3A to 3D and 4. FIG. 4 is a flow chart of a method for wearing a protective film of a physiological signal sensing device in accordance with the present invention.

The method of the embodiment in accordance with the present invention includes:

transporting an area (721) of the protective film (72) to a site of a moving path that a sensing module (20) moves toward an opening (12) by a transporter (S10);

fixing the area (721) at the opening (12) by the fastening (30) (S11);

moving the sensing module (20) through the opening (12) for stretching the area (721) over the sensing module (20) (S12).

By the steps (S10 to S12), a method for wearing a protective film over a sensing module (20) is accomplished.

Then the next steps are for next wearing process. The next steps include:

transporting an area (722) to the position of the area (721) located to replace the area (721) by a transporter (S20);

fixing the area (722) at the opening (12) by the fastening (S21);

moving the sensing module (20) through the opening (12) for stretching the area (722) over the sensing module (20). By repeat the steps described above, the process of wearing an area of a protective film can continue.

By means of the fastening (30) of the embodiment, the protective film (72) can properly fixed at the opening (12) and stretched over the sensing module (20). Hence, when users measure the ear temperature, the interference of measuring temperature caused by the unevenness of protective film can be solved.

Besides, the motion toward the opening (12) and rearwards away from the opening of the sensing module (20) drive the transporter (60) to transport protective film (72) from the containing portion (701) to the accepting portion (702) so as to not only move the used area of the protective film away from the opening to avoid second use but also efficiently use the moving distance of the sensing module for transporting an area of the protective film to the opening perfectly.

Although possible types of physiological signal sensing device and methods for wearing a protective film in accordance with the present invention have been described in the embodiment above, those skilled in the art shall recognized that different physiological signal sensing devices and methods for wearing a protective film can be designed. Therefore, the spirit of the present invention shall not be limit to these possible types of physiological signal sensing device and methods for wearing a protective film in accordance with the present invention. In other words, a device properly fix a protective film by a fastening and have the protective film stretched over an apparatus is the key spirit and scope of the present invention. The followings are some other embodiments in accordance with the present invention for those skilled in the art to know more about the spirit of the present invention.

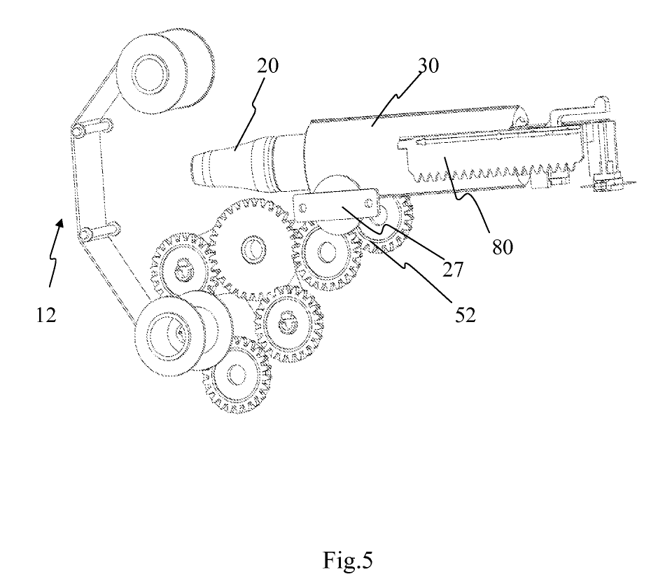

Please refer to both FIGS. 2A and 5, FIG. 5 is a schematic structure view of a second embodiment of a physiological signal sensing device in accordance with the present invention. The physiological signal sensing device of the embodiment further comprises a resistant (27) and a following (80) disposed at opposite side relative to the gear (52) and the follower (31) as in FIG. 2A. In another embodiment, the resistant (27) can be disposed at the same side as the gear (52) and the driving assembly (50) is shown in FIG. 2A and connect with the follower (31) referring to FIG. 2A so as to drive it by the follower (31). In this embodiment, when the sensing module (20) is moved toward the opening (12) till the following (80) is engaged with the resistant (27) and then the sensing module (20) keeps being moved, the resistant forms a resisting force against the motion force from the sensing module (20). Whereby, the effect of the motion force from the sensing module (20) can be cushioned. The resistant (27) can be designed as needed for providing a resisting force such as spring. In this embodiment, the resistant is a damper.

By the resisting force formed from the resistant (27), the motion force from the sensing module (20) can be cushioned effectively in case that the motion force of the sensing module (20) is too large to break the protective film while the sensing module (20) being moved though the opening (12).

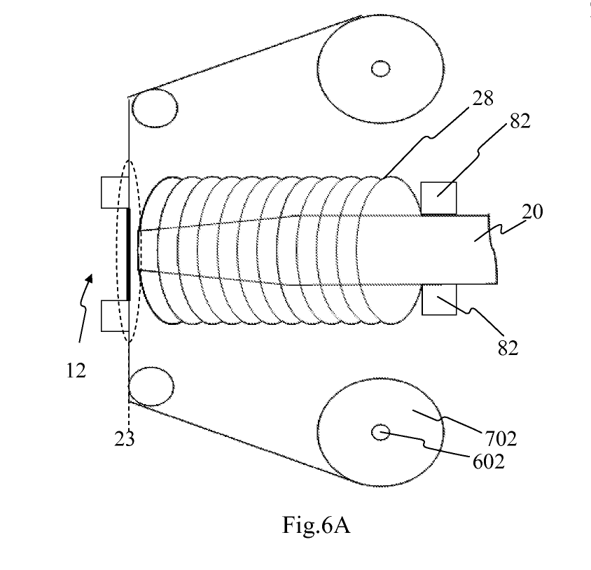

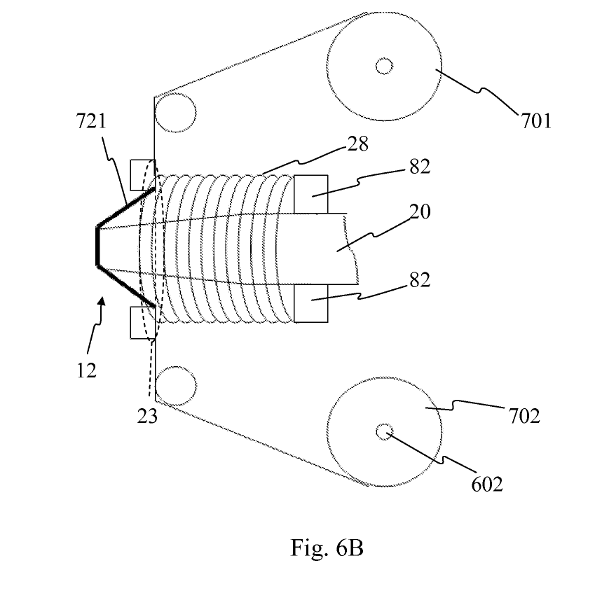

Please now refer to FIGS. 6A and 6B, FIG. 6A is a schematic structure view of a third embodiment of a physiological signal sensing device in accordance with the present invention showing that the sensing module is moved before a predetermined site. FIG. 6B is a schematic structure view of the physiological signal sensing device referring to FIG. 6A showing that the sensing module is moved over the predetermined site.

Please refer to FIGS. 2A, 5 and 6A. The physiological signal sensing device of this embodiment works similar to the first embodiment. The difference is that the resistant is an elastic member (28). The elastic member (28) connects with the following (82). In another embodiment, the following (82) can be designed as a part extended out from the sensing module (20). The elastic member (28) has both the function of the fastening (30) referring to FIG. 2A and the function of the resistant (27) referring to FIG. 5. The elastic member (28) is a spring surrounding the sensing module (20).

Referring to FIG. 6B, when the sensing module (20) is moved to a predetermined site (23) and keeps being moved over the predetermined site (23) and then moved through the opening (12). The elastic member (28) is compressed by the following (82) and forms an elastic potential energy to form a force to the area (721) so as to have the area (721) fixed at the opening (12). In addition, the elastic member (28) also forms a resisting force against the motion force of the sensing module (20) so as to cushion the motion force from the sensing module (20).

Therefore, by means of the elastic member (28) of the embodiment, the function of properly fixing the protective film, having the protective film evenly stretched over the sensing module and preventing the protective film from broken can be reached.

Please further referring to FIGS. 2C, 6A and 6B preferably, an immobilizing assembly referring to FIG. 2C is disposed as shown in this embodiment. When the protrusion (32) is coupled to the protrusion (33) to immobilizing the sensing module (20), the sensing module (20) is not moved back ward due to the resisting force of the elastic member. Whereby, the protective film can maintain the situation which evenly stretched over the sensing module (20) without keeping providing a force to the sensing module by users.

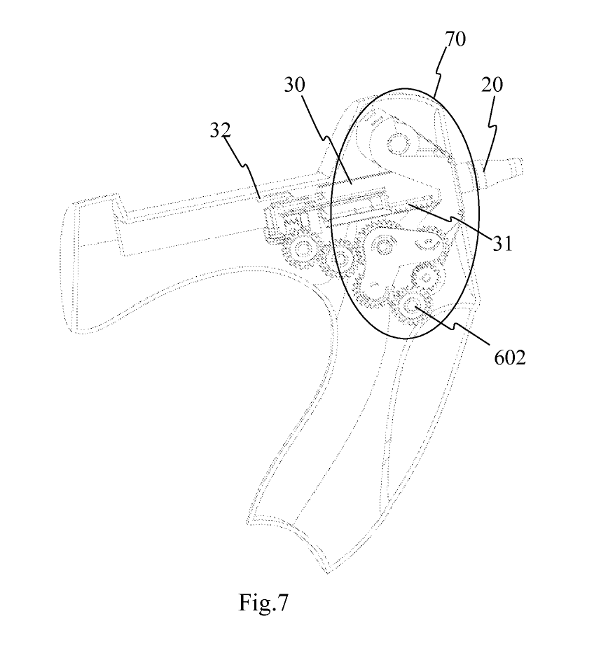

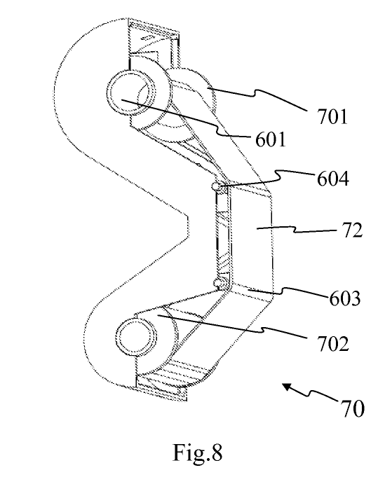

The protective-film containing assemblies in the embodiments described above are fixed within the physiological signal sensing device. However, the present invention should not be limited with it; the protective-film containing assembly can also be a detachable device. Please refer to FIGS. 7 and 8. FIG. 7 is a side perspective view of a forth embodiment of a physiological signal sensing device in accordance with the present invention. FIG. 8 is an enlargement view of a protective-film containing assembly referring to FIG. 7.

Please first refer to FIGS. 2A, 2C and 7, the physiological signal sensing device of the embodiment comprises a transporting member (602), a sensing module (20), a fastening (30), a driving assembly (50), a follower (31) and protrusions (32, 33) as in the first embodiment referring to FIGS. 2A and 2C. Referring to FIG. 7, the physiological signal sensing device of the embodiment further comprises a detachable protective-film containing assembly (70), which comprises a containing portion (701), an accepting portion (702) and transporting members (601, 603, 604). Comparing to the transporting device of the first embodiment, the transporting member (602) is fixedly disposed within the housing (10) in this embodiment. The transporting members (601, 603, 604) are disposed within the protective-film containing assembly (70). After the protective-film containing assembly (70) is equipped in the housing (10), the accepting portion (702) connects with the transporting member (602) so as to be driven by the sensing module (20), whose acting process as in the first embodiment. Users can easily change protective film by using the detachable protective-film containing assembly (70).

The sensing module is fixedly disposed within the housing in the above embodiments referring to FIGS. 2A, 5, 6A and 7. However, it can also be designed as a detachable one. Hence, the sensing module adapted to the specific physiological signal sensing device can be designed for sensing other physiological signal besides ear temperature. Users can pick up a specific sensing module for detecting specific physiological signal as needed. In the above embodiments, the fastening (30) or the elastic member (28) is disposed surround the sensing module. However, the fastening (30) or the elastic member (28) can be disposed in other way as needed such as disposed at one side or two sides of the sensing module.

In the above embodiment, the fastening (30) is driven by the sensing module (20). However, the fastening (30) can be designed as an independent device that operate independently. In an embodiment for example, the fastening (30) may be disposed at the side next to the opening and does not connect with the sensing module. When the fastening (30) is driven by a force, the protective film will be fixed by the fastening at the opening, whereas the sensing module is driven by another force and pass through the opening.

In the above embodiment, the sensing module (20) is an IR ear thermometer. However, it can also be designed as other sensing module that detect other physiological signals such as body temperature, forehead temperature, the size of pores in skin, humidity of skin or the image detected by ultrasonic system.

In the above embodiment of the present invention, a sensing module is disposed within the housing. In another embodiment, the sensing module of the physiological signal sensing device in accordance with the present invention can be replaced by a container for containing other member as needed such as different sensing module or a hard structure member and the whole device here is regarded as a containing device. For example, the container may contain a hard plastic structure member; the protective film may be designed for electrostatic dust collection. Whereby, this containing device can be used as a portable cleaning device for cleaning small and tiny places and the used part of the film can be changed with hand contacting.

Accordingly, the physiological signal sensing device or the containing device comprise a fastening which can have a protective film properly fixed and stretched over a sensing module or a container so as to prevent users from contact the protective film, in addition, the physiological signal sensing device in accordance with the present invention can prevent interference of sensing due to the protective film unevenly covers the sensing module. Furthermore, there are also other advantages in some embodiments of the present invention exemplarily listed as follows:

a. The physiological signal sensing device or the containing device in accordance with the present invention comprises a resistant which may prevent the protective film from breaking.

b. The fastening employed in the present invention designed as an elastic member may both fix the protective film and prevent it from breaking.

c. The physiological signal sensing device or containing device in accordance with the present invention comprises an immobilizing assembly which can make the protective film keep being stretched and evenly cover the sensing module or a container without users keeping providing force.

d. The physiological signal sensing device or containing device in accordance with the present invention comprises a driving assembly which may drive the transporter to transport the protective film by the motion of the sensing module.

Other embodiments of the invention will appear to those skilled in the art from consideration of the specification and practice of the invention disclosed herein. It is intended that the specification and examples to be considered as exemplary only, with a true scope and spirit of the invention being indicated by the following claims.

What is claimed is:

1. A physiological signal sensing device comprising:
a housing including an opening;
a sensing module engaged within said housing and moving through the opening for sensing a vital signal;
a transporter engaged within said housing for transporting an area of a protective film to a site corresponding to the opening, wherein said protective film has multiple areas; and
a fastening engaged within said housing for fixing said area of the protective film at said opening; when said sensing module moves toward said opening to a predetermined site, said fastening fixes said area at said opening; after said sensing apparatus keeps being moved through said opening, said area is stretched over said sensing module.

2. The physiological signal sensing device of claim 1, further comprising a resistant engaged with said sensing module, and when said sensing module is moved to the predetermined site, said resistant is driven by said sensing module for forming a resisting force against the motion of said sensing module.

3. The physiological signal sensing device of claim 1, further comprising an immobilizing assembly connected between said sensing module and said housing; after said area is stretched over said sensing module, said immobilizing assembly is fixed at a predetermined position to maintain a situation that said area is stretched over said sensing module.

4. The physiological signal sensing device of claim 1, further comprising a controller disposed on said sensing module and extended out from said housing for providing a holding area for moving said sensing module.

5. The physiological signal sensing device of claim 1, further comprising a protective-film containing assembly engaged with said transporter for containing said protective film.

6. The physiological signal sensing device of claim 1, further comprising a following connected between said sensing module and said fastening, and said following driven by the motion of said sensing module to drive said fastening to operate.

7. The physiological signal sensing device of claim 1, further comprising a driving assembly connected between said sensing module and said transporter, and driven by said sensing module to drive said transporter to transport said protective film.

8. The physiological signal sensing device of claim 7, further comprising a follower connected between said sensing module and said driving assembly for having said driving assembly be driven by the motion of said sensing module.

9. A containing device comprising:
a housing including an opening;
a container engaged within said housing and moving through the opening;
a transporter engaged within said housing for transporting an area of a protective film to a site corresponding to said opening, wherein said protective film has multiple areas; and
a fastening engaged within said housing for fixing said area at said opening; when said container is moved toward said opening to a predetermined site, said fastening fixes said area at said opening; after said container keeps being moved through said opening, said area is stretched over said container.

10. The containing device of claim 9, further comprising a resistant engaged with said container, and when said container is moved to the predetermined site, said resistant is driven by said container for forming a resisting force against the motion of said container.

11. A containing device for containing a protective film, which is adapted for a physiological signal sensing device comprising a housing, a sensing module, a transporter and a fastening, wherein said housing includes an opening, and said sensing module, said transporter and said fastening are engaged within said housing, said containing device comprising:
a containing portion for containing the protective film and engaged with said transporter, wherein said protective film has multiple areas, and said transporter is used for transporting an area of said protective film to a site corresponding to said opening; when said sensing module moves toward said opening to a predetermined site, said fastening fixes said area at said opening; after said sensing apparatus keeps being moved through said opening, said area is stretched over said sensing module.

12. The containing device of claim 11, further comprising an accepting portion for accepting said protective film from said containing portion.

13. A method for wearing a protective film, adapted for a physiological signal sensing device comprising a housing having an opening, a sensing module, a transporter and a fastening; said sensing module, said transporter and said fastening engaged within said housing, said method comprising:
transporting a first area of a protective film to a site corresponding to said opening by the transporter, wherein said protective film has multiple areas;
fixing said first area at said opening by the fastening;
moving said sensing module through said opening for stretching said first area over said sensing module;
transporting a second area of said protective-film to the position of said first area located so as to replace said first area by said transporter;
fixing said second area at said opening by said fastening; and
moving said sensing module through said opening for stretching said second area over said sensing module.
